(19) United States
(12) Patent Application Publication
(10) Pub. No.: US 2017/0066583 A1
(43) Pub. Date: Mar. 9, 2017

(54) System and Method For Packaging And Dispensing Disposable Garments

(71) Applicant: Brandon Kimbrough, Tempe, AZ (US)

(72) Inventor: Brandon Kimbrough, Tempe, AZ (US)

(21) Appl. No.: 14/849,293

(22) Filed: Sep. 9, 2015

(51) Int. Cl. B65D 83/08; A41D 13/12; A62B 17/00; A42B 1/04; A61K 33/18; A43B 3/02; A41D 27/20; B65H 16/00; A61K 9/70; B65D 85/672; A41D 19/015

(52) U.S. Cl. CPC B65D 83/08; B65D 85/672; A41D 13/1218; A62B 17/006; A42B 1/048; A41D 19/015; A43B 3/02; A41D 27/20; B65H 16/005; A61K 9/7023; A61K 33/18; A41D 2400/52; A41D 2400/70; B65H 2701/19

(57) ABSTRACT

A system for distributing garments at a point-of-use. The system includes a dispenser and a plurality of substantially planar sheets each comprising an individual garment disposed within and configured to be dispensed from the dispenser. Each garment includes a torso; first and second sleeves extending from the torso; a pocket disposed on a front portion of the torso; and a through-hole disposed in the pocket and configured to receive a wire therethrough.

### TECHNICAL FIELD

The present invention relates, generally, to biodegradable, recyclable perspiration garments and, more particularly, to improved methods for packaging them for distribution at their point of use.

### BRIEF SUMMARY

Various embodiments of the present invention relate to improved perspiration garments and methods for their manufacture, packaging, and distribution.

Other embodiments provide a biodegradable, recyclable, and/or disposable perspiration garment.

Other embodiments provide a perspiration garment with a pocket having a hole which accommodates an earplug wire extending from an electronic device held within the pocket.

Other embodiments provide a perspiration garment having tear lines which allow a user to remove portions of or the entire garment by ripping it along the tear lines.

Other embodiments provide a perspiration garment including integral coverings for the user's hands and feet extending from the shirt sleeves and pant legs, respectively.

Other embodiments provide a method of folding (e.g., pleating) a plurality of perspiration garments and/or disposable protective suits into a compact arrangement for distribution at an intended point of use. Those skilled in the art will appreciate that the folding and packaging techniques disclosed in the following publications, the entire contents of which are hereby incorporated herein by this reference, may employed in the context of the present invention: i) Chien et al. U.S. Publication No. US 2014/0317837; ii) Torres U.S. Publication No. US 2014/0123364; iii) White et al. U.S. Publication No. US 2014/0109282; iv) Wilbarger U.S. Publication No. US 2008/0256694; v) Smith U.S. Publication No. US 2006/0123530; vi) Bankemper U.S. Publication No. US 2003/0208836; and vii) Emanuel U.S. Publication No. US 2003/0061650.

Various other embodiments, aspects and features are of the present invention are described in more detail below. Additional features and characteristics will become apparent from the subsequent detailed description and the appended claims, taken in conjunction with the accompanying drawings and this background section.

### BRIEF DESCRIPTION OF THE DRAWING FIGURES

Exemplary embodiments will hereinafter be described in conjunction with the following drawing figures, wherein like numerals denote like elements, and:

FIG. 1 is a perspective view of a top portion of a perspiration garment including a pocket having an internal through hole configured to accommodate a headphone for an electronic device in accordance with exemplary embodiments of the present invention;

FIG. 2 is a perspective view of a top and bottom perspiration garment ensemble showing a headphone cord threaded through an internal through hole and extending internally from a pocket to the user's ear in accordance with exemplary embodiments of the present invention;

FIG. 3 is a perspective view of a top and bottom perspiration garment ensemble showing a headphone cord threaded through an external through hole and extending externally from a pocket to the user's ear in accordance with exemplary embodiments of the present invention;

FIG. 4 is a perspective view of a perspiration garment ensemble including a detachable hood in accordance with exemplary embodiments of the present invention;

FIG. 5 is a perspective view of a perspiration garment ensemble including integral gloves and boots in accordance with exemplary embodiments of the present invention;

FIG. 6 is a front elevation view of a perspiration garment showing an abdominal medical patch and various perforated tear lines in accordance with exemplary embodiments of the present invention;

FIG. 7 is a schematic view of a wall mounted point of use dispenser for perspiration garments in accordance with exemplary embodiments of the present invention;

FIG. 8 is a schematic view of an alternative mounting configuration for a point of use dispenser in accordance with exemplary embodiments of the present invention;

FIG. 9 is a perspective view of a roll of protective garments for point of distribution use in accordance with exemplary embodiments of the present invention;

FIG. 10 is a perspective view of a box of protective garments for point of distribution use in accordance with exemplary embodiments of the present invention;

FIG. 11 is a schematic front view of an alternative pocket configuration; and

FIG. 12 is a cross section view taken along line XII-XII of the pocket of FIG. 11.

### DETAILED DESCRIPTION OF PREFERRED EXEMPLARY EMBODIMENTS

The following detailed description is merely exemplary in nature and is not intended to limit the invention or the application and uses of the invention. Furthermore, there is no intention to be bound by any theory presented in the preceding background or the following detailed description.

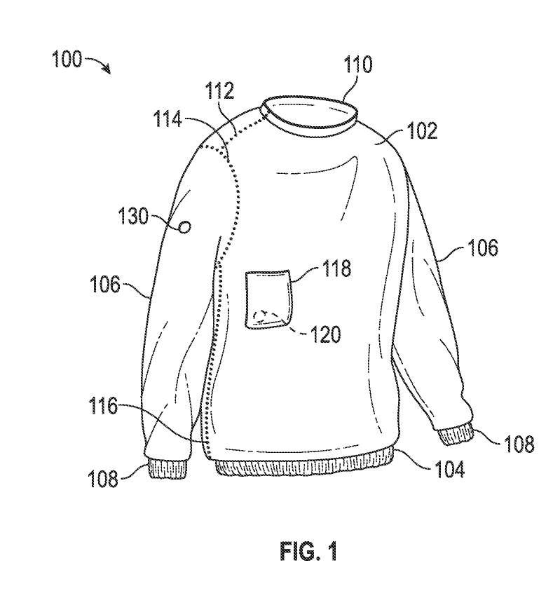

Referring now to FIG. 1, an exemplary perspiration garment 100 includes a torso portion 102, a suitable elastic or elastomeric waist band 104, sleeves 106 having suitable elastic or elastomeric wrist cuffs 108, respectively, an elastic or non-elastic collar 112, tear or perforation lines 112, 114, and 116, a pocket 118, optimally positioned proximate the bottom of the rib cage, the pocket including an internal hole 120, and an optional sleeve hole 30 for accommodating an internal ear plug wire extending from an arm band mounted device (not shown). Alternatively, the elastic or elastomeric components may comprise a draw string, Velcro, or any other suitable mechanism for closely forming the garment to the user's body.

In various embodiments, components such as sleeves, pant legs, hood, hand coverings, foot coverings, collar, waste band, wrist band, pocket, and the like, may be heat stamped to the garment assembly to avoid the cost associated with stitching. Alternatively, such components may be sewn, stitched, or otherwise secured to the torso in any desired manner.

In an embodiment, the garment material may be reusable and/or biodegradable. For example, a single or low multiple use garment may comprise a renewable bio-plastic or composite that decomposes into compost by the action living organisms (e.g., bacteria). Alternatively, a petrochemical-based material containing additives to enhance biodegradation may be employed to extend the useful life of the garment. Thus, by varying the material composition, thickness, and manufacturing processes, a perspiration garment may be produced for single use or for use in the range of 3-10 times before disposal.

With continued reference to FIG. 1, a material may be employed which has a protective layer applied to the internal and/or external surface of, or otherwise incorporated into the material itself. In this context, the protective layer may comprise an antimicrobial, anti-radiation, or other layer selected to protect the wearer from chemical, biological, biochemical, viral, bacterial, radiation, or other environmental agents.

The material may comprise any desired combination of color, texture, and surface finish features such as matte, reflective, metallic, two-tone, and/or multi-tone color schemes.

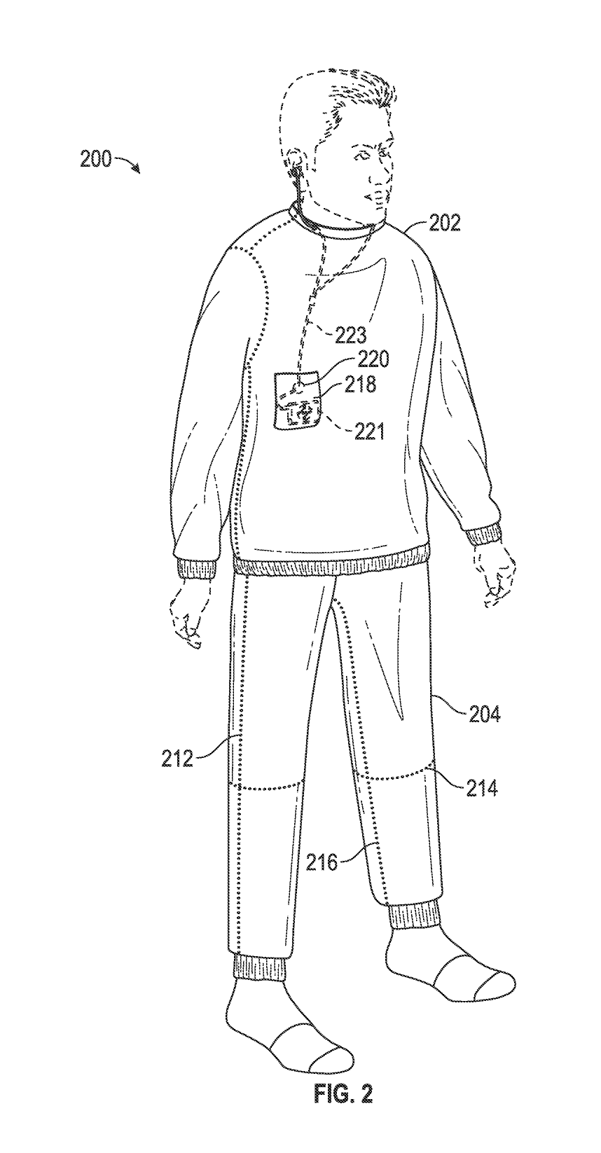

Referring now to FIG. 2, a perspiration/protective garment ensemble 200 includes a top portion 202 and a bottom portion 204. In the illustrated embodiment, the top portion 2002 includes a pocket 218 having an internal hole 220 for accommodating a headphone cord 223 extending internally (i.e., between the inside of the garment and the user's body) from an electronic device 221 to the user's ear. The bottom portion 204 may comprise a trouser or pant, including a trunk and respective leg coverings extending from the trunk.

Referring now to FIGS. 1 and 2, the garment may be form fitting, loose but comfortable, and may incorporate a degree of elasticity or stretchability to allow unencumbered movement during resistance and/or cardiovascular training. The material should also be sufficiently resilient to withstand rigorous physical activity without puncturing, tearing, or separating at the seams. In various embodiments, the garment may be configured to separate along one or more predetermined zones such as those defined by tear lines 112, 114, 116, 212, 214, 216. In this way, the user can remove a hood, collar, sleeves, pant leg, or even the entire garment without disrupting his or her workout, if desired.

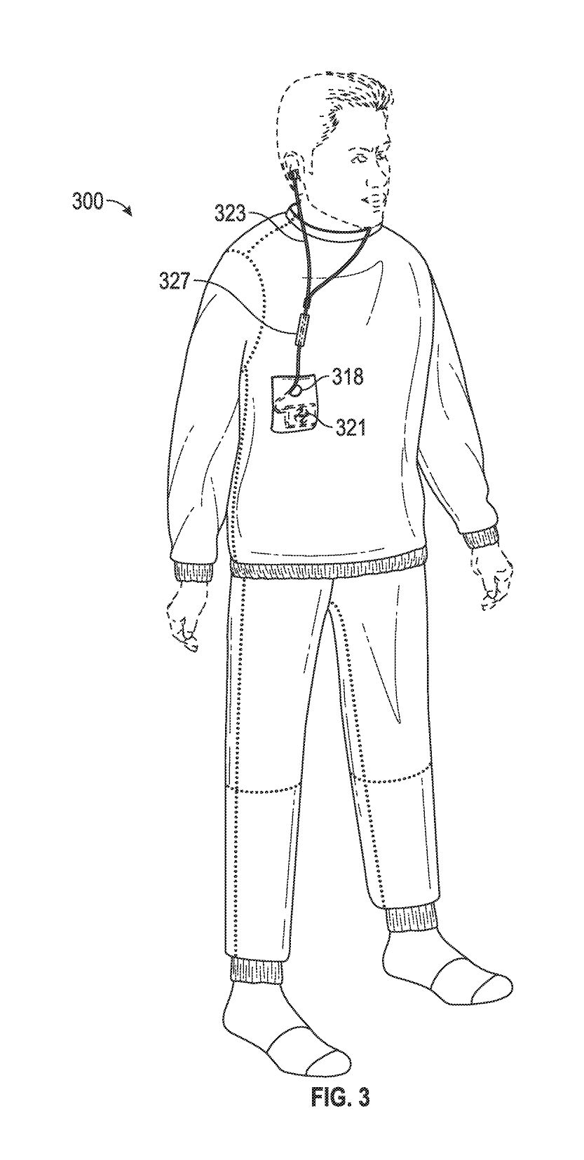

FIG. 3 is a garment ensemble 300 generally analogous to that shown in FIG. 2, including a headphone cord 323 threaded through an external through-hole 318 and extending externally from a pocket to the user's ear. As discussed in greater detail below, the head phone or ear plug device may connect to an electronic device such as a mobile phone, MP₃ player, IPod, land mobile radio (LMR), or other media or communications device.

In the embodiment shown in FIG. 2, the user may place the headphone or ear plug into or onto the ear before putting on the perspiration garment, such that the connecting wire extends internally between the user's body and the inside of the garment. In the embodiment shown in FIG. 3, the head phone device may be installed after putting on the garment, and worn external to the garment. If desired, the garment may include a support ring, loop, and/or or tube(s) 327 proximate the user's Adam's apple, breast, or solar plexus through which the connector 323 is threaded, to maintain the connector close to the user's body and safely away from moving persons, objects, or exercise equipment.

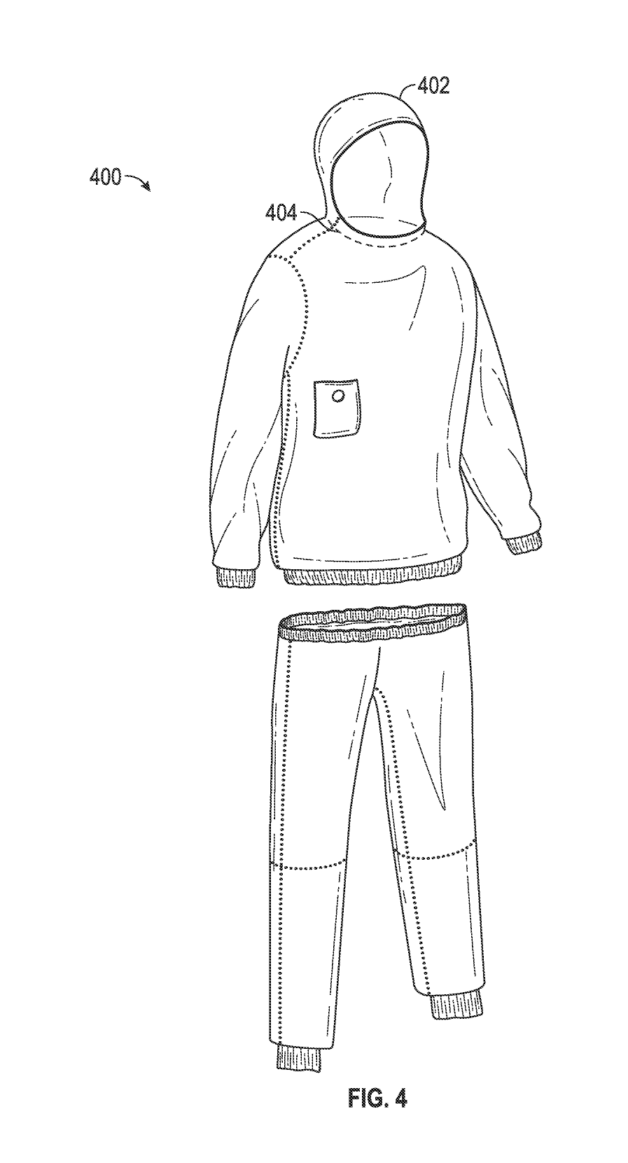

FIG. 4 is a perspiration/protective garment ensemble 400 including a detachable hood 402 and a perforation line 404 configured to facilitate the manual removal of the hood. In an embodiment, the hood also includes a transparent face mask (not shown) to protect the user from external contaminants.

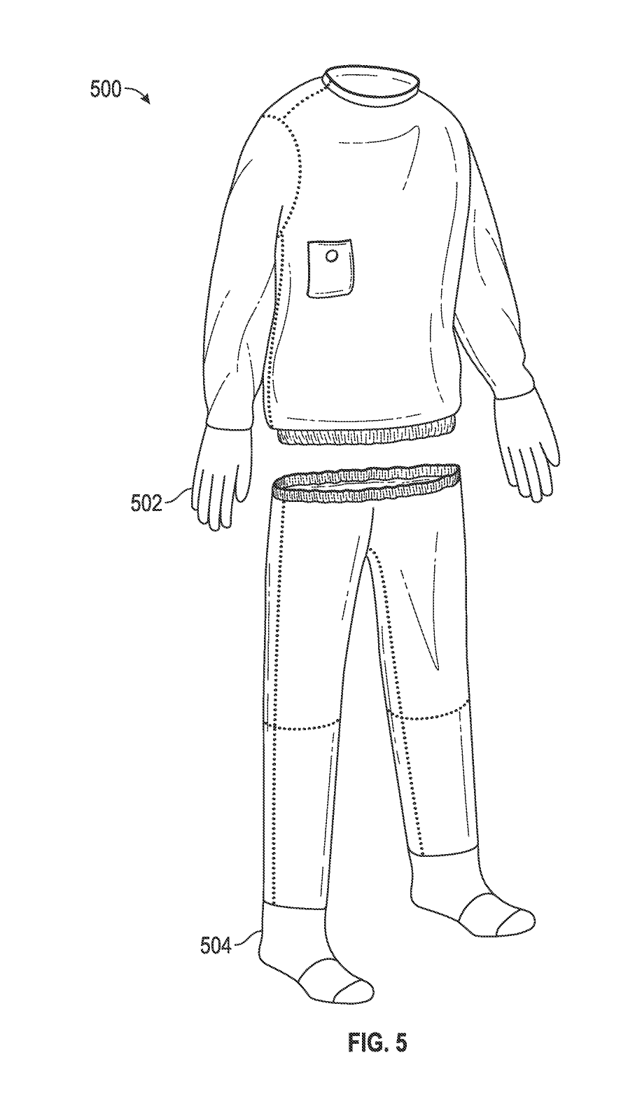

FIG. 5 is a perspiration and/or protective garment ensemble 500 for use in hazardous material (Hazmat), disaster relief, first responder, chemical and biohazard, quarantine, and hospital/surgical applications. The ensemble 500 may include integral gloves 502 and boots 504, as well as an optional hood and/or face mask (not shown). In the illustrated embodiment, the ensemble comprises a separate top and bottom component; alternatively, the ensemble may comprise a single piece garment having a zipper or other mechanism for sealing the garment around the user, and preventing or mitigating the introduction of external contaminants to the user's body.

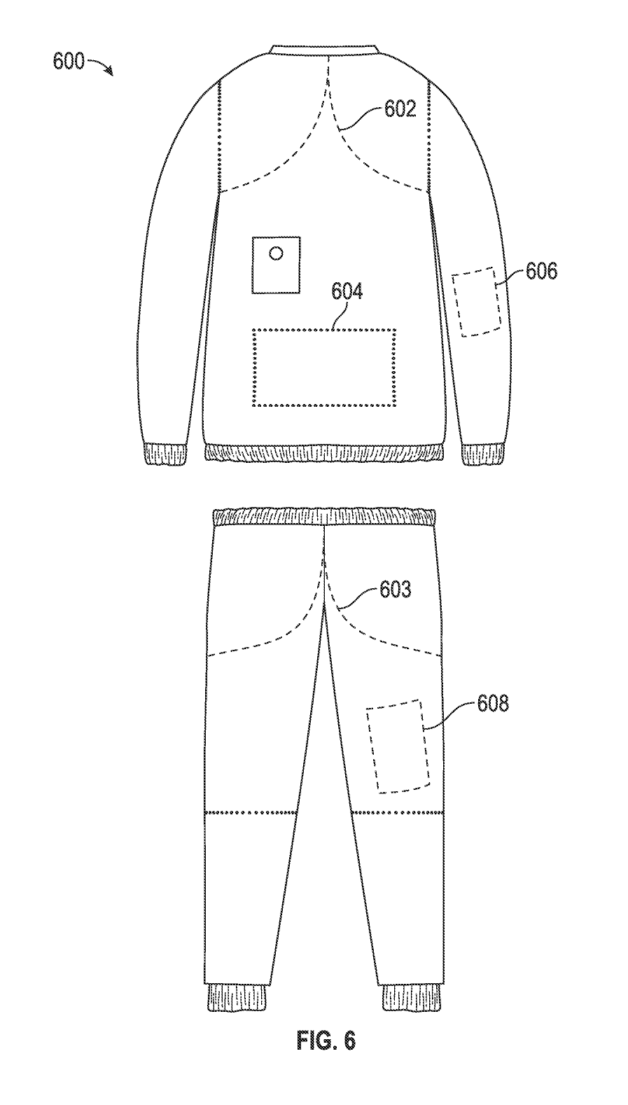

FIG. 6 is a front elevation view of a perspiration garment 600 having one or more topical, transcutaneous, and/or transdermal patches such as an abdominal (e.g., adhesive) patch 604, a forearm patch 606, and a thigh patch 608. The garment 600 may also comprise various perforated tear lines including pectoral lines 602 and groin/buttocks lines 603.

More particularly, the patches may comprise transdermal iodine such as an iodine tincture (e.g., 2% iodine solution, betadine, or providone) to mitigate radiation poisoning following a nuclear detonation, or any other medication or supplement to mitigate the effects of biological toxins or infectious agents such as revant, viruses, and fungi.

With continued reference to FIGS. 2-6, the various tear lines can be employed to facilitate the quick removal of all or a portion of the garment, either for safety reasons related to quarantine, or simply for the convenience and/or dramatic effect of ripping off one's clothing, for example, in a health club or work-out environment.

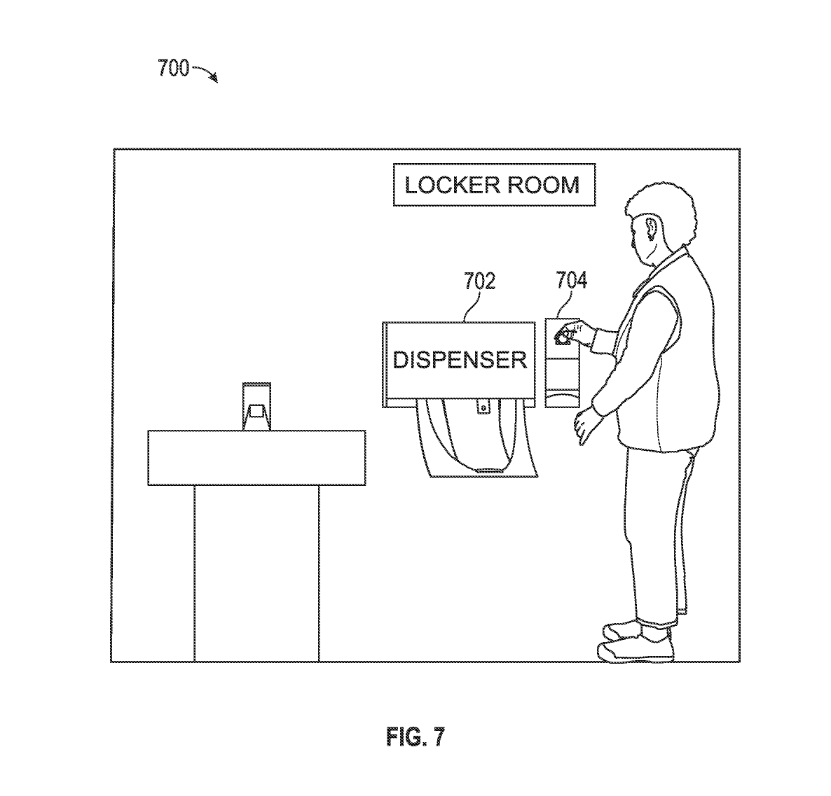

FIG. 7 is a schematic view of a dispensing system 700 including a wall-mounted point of use garment dispenser 702 and an optional interactive units 704 such as, for example, a magnetic stripe reader or point of sale (POS) terminal, coin slot, proximity sensor, smart card device, or the like. The dispensing system 700 may be placed anywhere that the garments are to be used, such as a health club, work-out facility, hospital, sports stadium, shopping mall, airport, first responder command post, or mobile dispensing device such as an aircraft, ground, or marine vehicle.

FIG. 8 is an alternative dispensing system 800 illustrating a table-top point of use dispensing apparatus 802 comprising a box of folded garments.

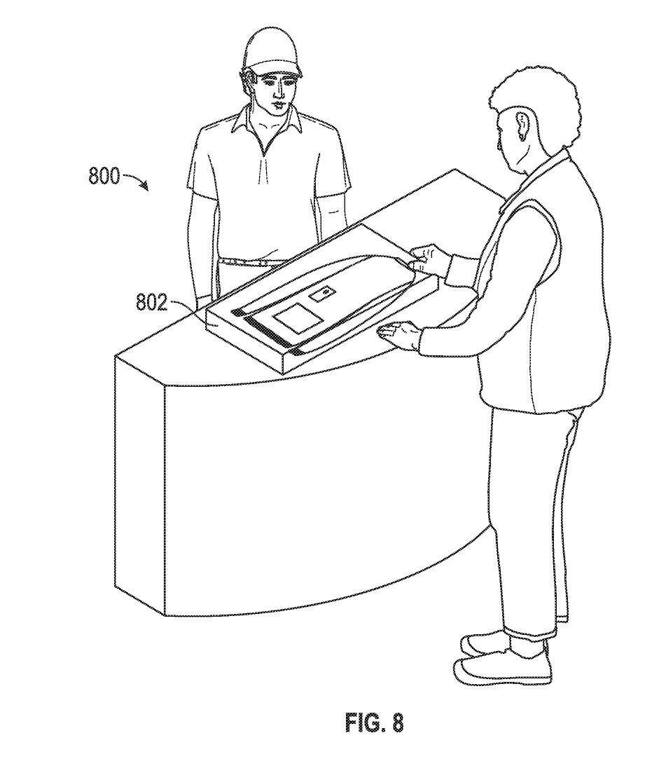

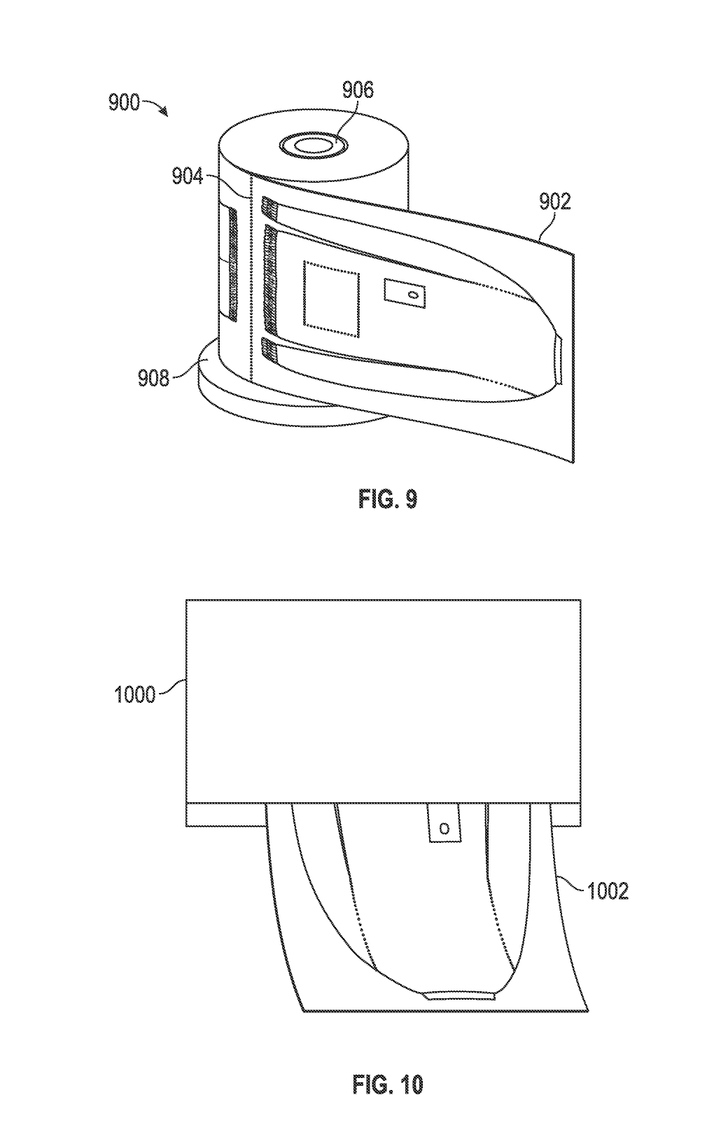

FIG. 9 is a roll 900 of protective garments for point of use distribution. In particular, the roll 900 comprises a plurality of sequential sheets 902 wound about a spool 908 and separated by perforations 904. In the illustrated embodiment, each sheet 902, when unfolded, comprises a top (torso) portion of a garment. Alternatively, each sheet 902 may comprise both a top and a bottom portion as well as optional gloves, boots, and a hood, as desired.

FIG. 10 is a box 1000 of protective/perspiration garments for point of use distribution. In particular, the box 1000 comprises a plurality of folded and/or pleated sheets 1002, with each sheet comprising either a top both a top and a bottom garment portion, as described above in connection with FIG. 9.

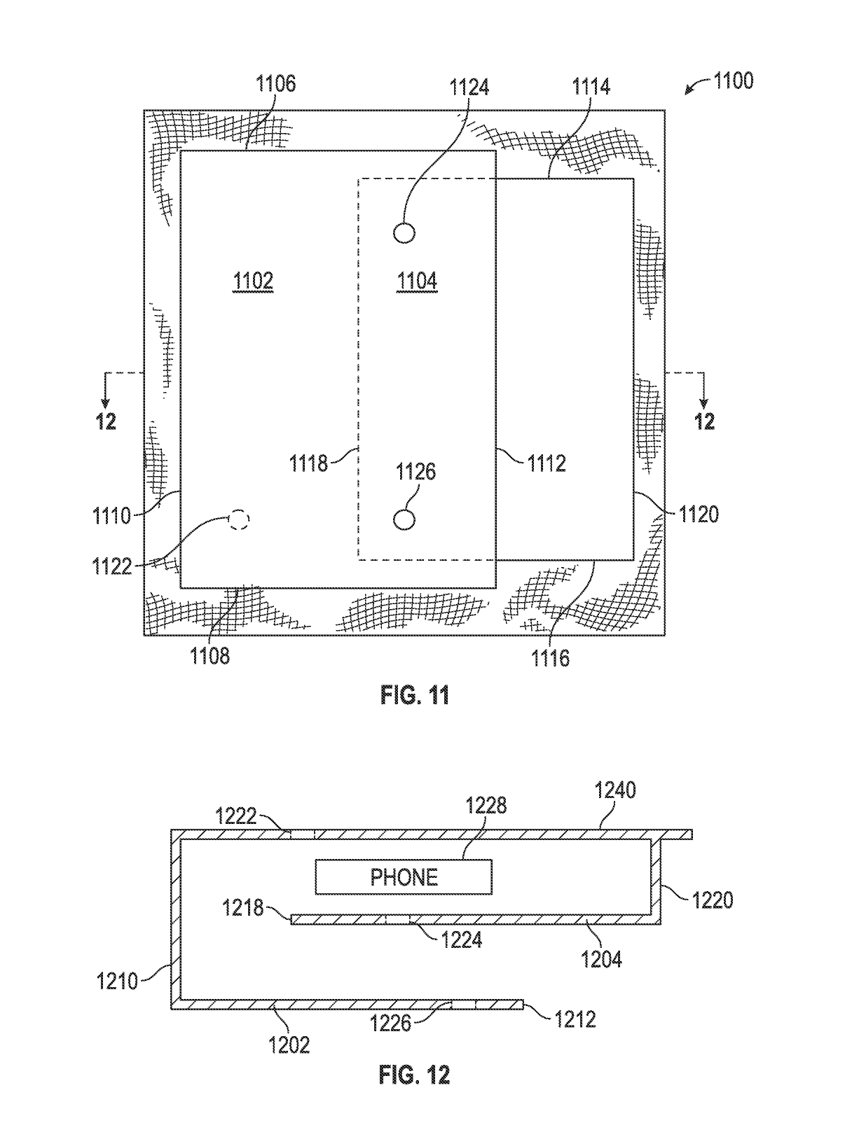

FIGS. 11 and 12 are schematic front elevation and cross section views, respectively, of an alternative pocket configuration 1100. In contrast to the embodiments shown in FIGS. 1-3 wherein the phone or communication device may be dropped into the pocket from above, the serpentine embodiment shown in FIGS. 11 and 12 facilitates inserting the device from the side, wherein it is secured at the top, bottom, left, and right pocket boundaries.

More particularly, the pocket assembly 1100 includes an outside panel 1102 partially overlapping an inside panel 1104. The outside panel 1102 includes a top edge 1106 and a bottom edge 1108, and the inside panel 1104 includes a top edge 1114 and a bottom edge 1116, with each edge forming a seam with the garment surface such that the device (e.g., media player) is vertically restrained. A first edge 1110 of the outside panel 1102, and a first edge 1120 of the inside panel 1104, also form a seam with the garment surface to horizontally restrain the device.

With continued reference to FIGS. 11 and 12, the user may first slide the device between panel 1102, 1202 and 1202, 1204, and thereafter into the space between panel 1104, 1204 and garment surface 1240 which is adjacent to user. An ear bud connector (not shown) can be configured to extend from the device to the user's ear through s hole located at position 1122, 1124 or 1126 in FIG. 11 (position 1222, 1224, or 1226 in FIG. 12).

A recyclable perspiration garment configured to be worn on a user's body is thus provided. The garment includes: a torso including a front portion and a back portion, each extending from a collar region to a waist band region; first and second sleeves extending from the torso; a pocket disposed on the front portion, the pocket having an open top and comprising an external pocket panel disposed parallel to and covering an internal pocket panel of the front portion, the external pocket panel forming a left edge seam, a right edge seam, and a bottom seam with the internal pocket panel; a through-hole disposed in the pocket and configured to receive a wire therethrough; and at least one perforation line configured to facilitate the manual tearing and removal of at least a portion of the garment from the user's body.

In an embodiment, the perforation line is disposed in a pectoral region of the front portion.

In an embodiment, the first and second sleeves are connected to the torso along respective sleeve perforation lines.

In an embodiment, the through-hole is disposed in at least one of the internal pocket panel and the external pocket panel.

In an embodiment, the garment further includes a wire restraint connected to the front portion between the collar region and the pocket.

In an embodiment, the garment further includes a second through-hole disposed in an upper region of one of the sleeves and configured to receive a wire therethrough.

In an embodiment, the garment further includes a trouser including a second perforation line configured to facilitate the manual tearing and removal of at least a portion of the trouser from the user's body.

In an embodiment, the trouser comprises first and second leg coverings connected to a trunk along respective trunk perforation lines.

In an embodiment, the garment further includes at least one of: a hood connected to the collar region; respective gloves extending from the distal end of each sleeve; and respective boots extending from the distal end of each leg covering.

In an embodiment, the garment further includes a transdermal patch, which may be disposed proximate at least one of an abdominal, forearm, or thigh region.

A disposable protective garment configured to be worn on a user's body is also provided. The garment includes a torso including a front portion and a back portion, each extending from a collar region to a waist band region; first and second sleeves extending from the torso; a pocket disposed on the front portion, the pocket comprising an outside panel at least partially overlapping an inside panel, the outside and inside panels each comprising respective horizontally extending top and bottom seams with the front portion, the outside and inside panels each further comprising a vertically extending seam with the front portion; and a through-hole disposed in the pocket and configured to receive a wire therethrough.

In an embodiment, the through-hole is disposed in at least one of the inside panel, the outside panel, and the front portion covered by the pocket.

In an embodiment, the garment further includes a trouser having first and second leg segments connected to a trunk.

In an embodiment, the garment further includes tear lines configured to facilitate the manual removal of at least a portion of the garment from the user's body.

In an embodiment, the garment t further includes: a hood connected to the collar region; respective gloves extending from the distal end of each sleeve; and respective boots extending from the distal end of each leg segment.

In an embodiment, the garment further includes a transdermal patch proximate at least one of an abdominal, forearm, or thigh region.

A system is also provided for distributing garments at a point-of-use. The system includes a dispenser and a plurality of substantially planar sheets each comprising an individual garment disposed within and configured to be dispensed from the dispenser. Each garment includes: a torso; first and second sleeves extending from the torso; a pocket disposed on a front portion of the torso; and a through-hole disposed in the pocket and configured to receive a wire therethrough.

In an embodiment, the dispenser comprises one of: a box within which the planar sheets are stacked on top of and parallel to one another; and a spool about which the planar sheets are wound, the planar sheets being separated by tear lines.

In an embodiment, the pocket comprises one of: an open top and an external pocket panel disposed parallel to and covering an internal pocket panel region of the torso, the external pocket panel forming a left edge seam, a right edge seam, and a bottom seam with the internal pocket region of the torso; and an outside panel at least partially overlapping an inside panel, the outside and inside panels each comprising respective horizontally extending top and bottom seams, the outside and inside panels each further comprising a vertically extending seam.

In an embodiment, the system further includes a transdermal patch proximate at least one of an abdominal, forearm, or thigh region.

While the foregoing detailed description will provide those skilled in the art with a convenient road map for implementing various embodiments of the invention, it should be appreciated that the particular embodiments described above are only examples, and are not intended to limit the scope, applicability, or configuration of the invention in any way. To the contrary, various changes may be made in the function and arrangement of elements described without departing from the scope of the invention

What is claimed:

1. A recyclable perspiration garment configured to be worn on a user's body, comprising:
a torso including a front portion and a back portion, each extending from a collar region to a waist band region;
first and second sleeves extending from the torso;
a pocket disposed on the front portion, the pocket having an open top and comprising an external pocket panel disposed parallel to and covering an internal pocket panel of the front portion, the external pocket panel forming a left edge seam, a right edge seam, and a bottom seam with the internal pocket panel;
a through-hole disposed in the pocket and configured to receive a wire therethrough; and
at least one perforation line configured to facilitate the manual tearing and removal of at least a portion of the garment from the user's body.

2. The garment of claim 1, wherein the perforation line is disposed in a pectoral region of the front portion.

3. The garment of claim 1, wherein the first and second sleeves are connected to the torso along respective sleeve perforation lines.

3. The garment of claim 1, wherein the through-hole is disposed in at least one of the internal pocket panel and the external pocket panel.

4. The garment of claim 1, further comprising a wire restraint connected to the front portion between the collar region and the pocket.

5. The garment of claim 1, further comprising: a second through-hole disposed in an upper region of one of the sleeves and configured to receive a wire therethrough.

6. The garment of claim 1, further comprising:
a trouser including a second perforation line configured to facilitate the manual tearing and removal of at least a portion of the trouser from the user's body.

7. The garment of claim 6, wherein the trouser comprises first and second leg coverings connected to a trunk along respective trunk perforation lines.

8. The garment of claim 6, further comprising at least one of:
a hood connected to the collar region;
respective gloves extending from the distal end of each sleeve; and
respective boots extending from the distal end of each leg covering.

9. The garment of claim 6, further comprising a transdermal patch.

10. The garment of claim 9, wherein the patch is disposed proximate at least one of an abdominal, forearm, or thigh region.

11. A disposable protective garment configured to be worn on a user's body, comprising:
a torso including a front portion and a back portion, each extending from a collar region to a waist band region;
first and second sleeves extending from the torso;
a pocket disposed on the front portion, the pocket comprising an outside panel at least partially overlapping an inside panel, the outside and inside panels each comprising respective horizontally extending top and bottom seams with the front portion, the outside and inside panels each further comprising a vertically extending seam with the front portion; and
a through-hole disposed in the pocket and configured to receive a wire therethrough.

12. The garment of claim 11, wherein the through-hole is disposed in at least one of the inside panel, the outside panel, and the front portion covered by the pocket.

13. The garment of claim 11, further comprising a trouser having first and second leg segments connected to a trunk.

14. The garment of claim 11, further comprising tear lines configured to facilitate the manual removal of at least a portion of the garment from the user's body.

15. The garment of claim 14, further comprising:
a hood connected to the collar region;
respective gloves extending from the distal end of each sleeve; and
respective boots extending from the distal end of each leg segment.

16. The garment of claim 11, further comprising a transdermal patch proximate at least one of an abdominal, forearm, or thigh region.

17. A system for distributing garments at a point-of-use, comprising:
a dispenser; and
a plurality of substantially planar sheets each comprising an individual garment disposed within and configured to be dispensed from the dispenser, each garment comprising:
a torso;
first and second sleeves extending from the torso;
a pocket disposed on a front portion of the torso; and
a through-hole disposed in the pocket and configured to receive a wire therethrough.

18. The system of claim 17, wherein the dispenser comprises one of:
a box within which the planar sheets are stacked on top of and parallel to one another; and
a spool about which the planar sheets are wound, the planar sheets being separated by tear lines.

19. The system of claim 17, wherein the pocket comprises one of:
an open top and an external pocket panel disposed parallel to and covering an internal pocket panel region of the torso, the external pocket panel forming a left edge seam, a right edge seam, and a bottom seam with the internal pocket region of the torso; and
an outside panel at least partially overlapping an inside panel, the outside and inside panels each comprising respective horizontally extending top and bottom seams, the outside and inside panels each further comprising a vertically extending seam.

20. The system of claim 17, further comprising a transdermal patch proximate at least one of an abdominal, forearm, or thigh region.
